(19) United States
(12) Patent Application Publication
(10) Pub. No.: US 2003/0171451 A1
(43) Pub. Date: Sep. 11, 2003

(54) Bony tissue fillers and restoratives containing biocompatible particle

(76) Inventors: Daniel A. White, Minnetonka, MN (US); Dean A. Klein, North Oaks, MN (US); James D. Brazil, Braham, MN (US)

Correspondence Address: FAEGRE & BENSON LLP, 2200 WELLS FARGO CENTER, 90 SOUTH 7TH STREET, MINNEAPOLIS, MN, 55402, US

(21) Appl. No.: 10/095,333

(22) Filed: Mar. 11, 2002

(51) Int. Cl. A61K 6/08; C08K 3/18; C08K 3/04

(52) U.S. Cl. 523/117; 524/430; 524/495

(57) ABSTRACT

This invention provides biocompatible, load bearing compositions adapted for bone filling and restorative applications or purposes. One embodiment of this invention includes curable compositions comprising carbon coated composite particles in a polymerizable mixture. Another comprises carbon coated composite particles combined with autogenous bone. These mixtures may be adhered to metals, ceramics, and natural bone tissues. Embodiments of this invention are highly compatible with natural bone tissue and are capable of bearing significant loads.

### BACKGROUND

The demand for biomaterials that are suitable for use as grafting, filling or restorative materials in orthopedic and dental applications continues to increase. A significant amount of research into biomaterials for orthopedic and dental applications has focused on the requirements that suitable biomaterial grafts, fillers or restoratives attach and form immediately to a particular site in the body, bond strongly to bone or bony tissue, and provide strong, highly resilient structures.

Autologous bone has been traditionally used as a bone filler or restorative. Autologous bone is bone tissue harvested from a donor site on a patient and applied to a diseased or injured site on that patient. Autologous bone is a suitable bone filler because it provides scaffolding for osteoconduction, growth factors for osteoinduction and progenitor stem cells for osteogenesis. However, there are several drawbacks to autologous bone, including limited availability, increased operation time and donor site morbidity. Thus, biocompatible bone substitutes have been used as an alternative to autologous bone fillers or restoratives.

Conventional biomaterials that have been used as orthopedic and dental fillers or restoratives are commonly bone cements that are based on acrylate-containing compositions such as polymethyl methacrylate (PMMA) cements or compositions. These PMMA cements or compositions are typically capable of convenient delivery to a targeted body site and have reasonable degrees of affinity for bone or bony tissue. PMMA cements or compositions, however, are limited as to strength and lack both bioactivity and an ability to generate formation of bony tissue. Further, the inertness of PMMA cements may lead to micromotion and fatigue over time. Still further, the polymerization process of PMMA cements in the body may generate significant amounts of heat that may lead to localized tissue necrosis and inflammation. Moreover, residual methyl methacrylate monomer contained in PMMA cements may leach into surrounding tissue and lead to inflammation and an unacceptable result.

Other biomaterials that have been used as orthopedic or dental fillers or restoratives include bioactive glasses, collagen, or mixtures of these materials. These materials generally have good biocompatibility and may lead to bony tissue formation but these materials often lack desirable load bearing strength. Still other materials include calcium phosphate cements as well as glass ionomer bone cements. Both of these types of materials may be bioactive and exhibit suitable strength. Glass ionomer bone cements, in particular, have been used successfully in dental applications. Yet other biocompatible materials include different glasses, glass-ceramics, and crystalline phase materials, either alone or in combination with acrylate polymers or other acceptable polymers. Exemplary types of glass or ceramic materials include hydroxyapatite, fluorapatite, oxyapatite, Wollastonite, anorthite, calcium fluoride, agrellite, devitrite, canasite, phlogopite, monetite, brushite, octocalcium phosphate, Whitlockite, tetracalcium phosphate, cordierite, and Berlinite.

A need exists for suitable biocompatible materials that provide a bone filler or restorative composite that is strong and tissue compatible.

### SUMMARY OF THE INVENTION

One embodiment of the present invention provides a curable composition that contains two primary components. A first component is biocompatible detectable composite particles having an exposed surface of carbon. A second component is a polymerizable mixture. Another embodiment of the invention provides a composition that contains autogenous bone and biocompatible detectable composite particles having an exposed surface of carbon.

In certain embodiments, the particles are isotropic pyrolytic carbon coated zirconia oxide or carbon substrates. Suitable particles, in some embodiments, have a transverse cross-section dimension of about 10-300 microns. Alternative embodiments include particles having a transverse cross-section dimension of about 50-120 microns. In use, compositions of this invention may include about 10-40 wt. % composite particles. The particles may also be radiopaque.

A suitable polymerizable mixture used in curable embodiments of the present invention includes curable acrylate monomer(s) and catalyst(s) systems such as polymethylmethacrylate/catalyst systems. A variety of polymerizable monomers, oligomers and/or reactive polymers have been used in known filling or restorative formulations. These known polymerizable mixtures may be used in selected embodiments of the present invention. For example, a variety of a polymerizable acrylates may be used in the polymerizable mixture including, but not limited to, bisphenol-A derived acrylates such as bisphenol-A dimethacrylate and bisphenol-A glycidyl dimethacrylate.

Catalysts or catalytic agents and/or systems used to aid polymerization of suitable mixtures are also widely known and any of these known catalytic agents and/or catalytic systems may be employed in the present invention. In general, “thermal” and “photopolymerizable” systems may be used. A number of peroxides, such as benzyl peroxide are used in heat curable formulations. In addition, photopolymerization systems are also useful, especially when the curing is done with visible light. For example, camphoroquinone, especially when mixed with a tertiary amine, is a known visible light curing catalyst.

Autogenous bone used in embodiments of the present invention may be suitable for bone filling or restoring because it possesses excellent growth characteristics when applied to an injured or diseased bony tissue location. As used herein, “autogenous bone” refers to bone harvested from one or more donor or donor sites that is suitable for application to a patient. For example, autogenous bone may be harvested from one or more cadavers and then processed for use as a bone filler. Biocompatible detectable particles mixed with the autogenous bone may provide improved bone ingrowth and scaffolding, as well as increased mechanical strength at the application site.

One embodiment of the present invention provides a method for bony tissue filling or restoration. The principle steps of this method are applying a curable composition made of biocompatible detectable composite particles having an exposed surface of carbon and a polymerizable mixture to bony tissue and then curing the polymerizable mixture to provide a load bearing, cured or hardened matrix. In particular, an area of bony tissue requiring repair as a result of disease, injury, or desired reconfiguration is generally surgically prepared and a composition of the present invention applied or introduced into the prepared site. The composition is then cured, hardened or polymerized through either heat or photochemistry, the wound closed, and the repaired site is allowed to heal.

Another embodiment of the present invention provides a method for bony tissue filling or restoring wherein a composition including biocompatible detectable particles having an exposed surface of carbon and autogenous bone is applied to a patient site. In particular, an area of bony tissue requiring repair as a result of disease, injury, or desired reconfiguration is generally surgically prepared and a bone filler or restorative of the present invention is injected into the prepared site.

A variety of application processes are suitable, depending on the type of composition or filler and the particular procedure being used. An example of a suitable application process includes injecting a composition of the present invention at, onto and/or into the prepared site. Optionally, the injection may be percutaneous. One characteristic of injecting the composition into a diseased site is that the patient is subjected to a minimally invasive surgical procedure. For this exemplary application process, viscosities for the curable compositions of this invention range between about 5,000-75,000 centipoise.

### DETAILED DESCRIPTION

This invention provides biocompatible, load bearing, compositions well suited for bone filling and restorative applications or purposes. One embodiment of this invention is a curable composition comprising detectable carbon coated composite particles in a polymerizable mixture. Another embodiment of the present invention provides a composition including autogenous bone and biocompatible detectable particles having exposed surfaces of carbon. Representative embodiments of the present invention may be applied to metals, ceramics and natural bone tissues. Selected embodiments of this invention are highly compatible with natural bone tissue and capable of bearing significant loads.

The particles of the present invention are generally durable, stable and hard, and comprise metallic, ceramic or carbon substances. The particles are also preferably radiopaque. Aluminum oxide and zirconium oxide are both suitable particles. Other particles such as metallic particles, including but not limited to, medical grade stainless steel, titanium and titanium alloys and all oxide derivatives of each, are also acceptable. Graphite may also be utilized as a satisfactory, low cost particle.

In select compositions of the present invention, the particles comprise a generally durable, stable and hard substrate that has a thin coating or film of biocompatible carbon deposited on the substrate's outer exposed surfaces. Suitable substrates include ceramic, metallic or carbon substrates. In one embodiment, for example, the particles are carbon coated zirconium or aluminum oxide substrates. In another embodiment, a non-pyrolytic carbon substrate is coated with isotropic pyrolytic carbon. In yet another embodiment, a total pyrolytic carbon particle may comprise the particle. Thus, the beads in some embodiments may be comprised entirely of carbon.

Different types of carbon coating processes may be utilized provided the substrate is a material that is selected for compatibility with the coating process. Particles are typically completely encased by a thin carbon coating that provides a smooth coated particle with no substrate exposure on the surface of the particle or in contact with tissue when used.

Low temperature isotropic (LTI) pyrolytic carbon is an exemplary carbon coating. Pyrolytic carbon is produced in a process in which hydrocarbons and alloying gases are decomposed in a fluidized or floating bed of a desired substrate. Inert gas flow is used to float the bed and the substrate particles. The hydrocarbon pyrolysis results in spheres having a high carbon atom, low hydrogen atom content that deposit on the accessible surfaces of the substrate in the fluidized bed. As the spheres deposit at temperatures of 1200°-1500° C., they may coalesce, deform or grow, resulting in a high density carbon coating on the substrate surface.

Ultra-low-temperature isotropic carbon may be also be applied as a coating in vacuum vapor deposition processes. A carbon coating may be deposited effectively utilizing ion beams generated from the disassociation of CO₂, reactive disassociation in vacuum of a hydrocarbon as a result of a glow discharge, or sublimation of a solid graphite source or cathode sputtering of a graphite source. Gold has been found to be suitable as a substrate for vacuum vapor deposited carbon, however, other substrates including but not limited to, nickel, silver, stainless steel, or titanium are also acceptable.

Vitreous or glass carbons may also serve as the coating material. These are also isotropic, monolithic carbons, which are formed by pyrolysis of carbonaceous preforms, during which gaseous pyrolysis products diffuse through the shape.

The atomic structure of either LTI pyrolytic carbon or vitreous carbon is similar to graphite, the common form of carbon, but the alignment between hexagonal planes of atoms is not as well ordered. Pyrolitic carbon is characterized by a more chaotic atomic structure with warped hexagonal planes, missing atoms and generally a more turbostatic appearance. This results in better bonding between layer planes.

The coating process is applied to small substrate particles to produce final, rounded particles that have a smooth carbon-coated surface in the form of a thin film. The resulting smooth surface on the particles enhances their passage through an injection needle, cannula or catheter and into selected body sites. The high strength, resistance to breakdown or corrosion, and durability of the carbon coating insures the effective, long term functioning of the particles at the site. The established biocompatibility of pyrolytic carbon renders it particularly suitable for internal body applications.

After the carbon coating has been applied, the particles are subjected to a cleaning and sieving process to remove contaminants and to separate out particles of a size less than or greater than the desired size range. Typically the particles range in size from 10 microns to 1,000 microns in average, transverse cross-sectional dimension, and a suitable size range is between 10 and 300 microns. A size that allows injection through a small bore instrument may be used in some applications. The substrate particles are initially milled, extruded or otherwise formed to the desired particle size, in a substantially rounded shape prior to being subjected to the coating process. The particles are randomly shaped and rounded, ranging from oblong to generally spherical. The sieving process is such that the minimum particle dimension will pass through a U.S. No. 18 Screen Mesh (1000 micron grid size opening) but will not pass through a U.S. No. 140 Screen Mesh (106 micron grid size). That minimum dimension will be the transverse, cross-sectional dimension on oblong or elongated particles, with that dimension coinciding with the particle diameter on generally spherical particles.

Polymerizable mixtures suitable for use in the practice of one or more embodiments of the present invention include a variety of ethylenically unsaturated and other known polymerizable compositions or mixtures. Acrylic and acrylate compositions are suitable. These mixtures may be selected from the class of acrylate polyesters. For example, the bis-glycidyl methacrylate adduct of bisphenol-A (bis-GMA) and its other related acrylate mixtures are suitable. Alternatively, adducts of 2,2,3-trimethylhexane diisocyanate with hydroxyethyl methacrylate, hydroxypropyl methacrylate, and other hydroxyacrylate compositions are also suitable. Those of ordinary skill in the art will appreciate that other acrylate polyesters may also be suited for use and that these mixtures may be reacted with isocyanates to form urethanes useful as polymerizable compositions or mixtures. For example, bis-GMA may be reacted with diisocyanate or other isocyanates such as hexamethylene diisocyanate, phenylene diisocyanate or a variety of other aliphatic and aromatic diisocyanates to provide useful polymerizable compositions or mixtures. Adducts of bis-GMA hexamethylene diisocyanate may also be useful for polymerizable mixtures of the present invention.

Methyl methacrylate, ethyl methacrylate, propyl methacrylate, and higher methacrylates, acrylates, ethacrylates, as well as mixtures of these monomers may be used as all or part of the polymerizable mixture of the present invention. It is also suitable to use other polymerizable mixtures such as epoxide monomers, polyurethane-precursor monomers as well as other polymerizable monomers or materials. For example, other monomers useful in the production of polymerizable mixtures of this invention include isopropylacrylate, tert-butyloctylacrylate, dodecylacrylate, cyclohexylacrylate, chloromethylacrylate, tetrachloroethylacrylate, perfluorooctylacrylate hydroxyethylacrylate, hydroxypropylacrylate, hydroxybutylacrylate, 3-hydroxyphenylacrylate, 4-hydroxphenylacrylate, aminoethylacrylate, aminophenylacrylate, and thiophenylacrylate, acrylate, methacrylate, ethacrylate, propacrylate, butacrylate and chloromethacrylate, as well as the homologous mono-acrylic acid esters of bisphenol-A, dihydroxydiphenyl sulfone, dihydroxydiphenyl ether, dihydroxybiphenyl, dihydroxydiphenyl sulfoxide, and 2,2 bis(4-hydroxy-2,3,5,6-tetrafluorophenyl)propane. Polymerizable monomers capable of sustaining a polymerization reaction such as the di-, tri-, and higher acrylic ethylene glycol dimethacrylate, diethylene glycol dimethacrylate, trimethylene glycol dimethacrylate, trimethylol propane trimethacrylate, analogous acrylates, and similar monomers, oligomers or polymers are also useful. It is also suitable to employ mixtures of two, three, and more polymerizable monomers, oligomers or polymers.

Bis-GMA-based polymerization systems are particularly useful in restorations within the human body. These systems are generally biocompatible, are free of significant amounts of toxic products, are similar in physical characteristics to bony tissue (such as compatible coefficients of thermal expansion), are relatively easy of use, and are stable.

It is generally necessary to provide catalysis for the polymerizable mixtures used in the curable or hardenable compositions of the present invention. Such catalysts are typically of two general classes, thermal and photopolymerization catalysts or initiators. Each class is well-known and any catalytic system known for restorative use may be employed so long as the system provides a suitable product when used.

Thermal or heat curing catalysts or initiators are generally used in “two-paste” systems. In this catalytic system, two components of the curable or hardenable composition are mixed together, the catalytic action begins, leading to curing or hardening. This system may be applied to a wide variety of polymerizable mixtures that are suitable in the present invention. Radical initiators such as peroxides, especially benzoyl peroxide (also called dibenzoyl peroxide) are conventional, economic, and convenient. A stabilizer, such as butyl hydroxytoluene is often used. Use of co-catalysts such as dimethyl-p-toluidine, N-N-substituted toluidine and oxylidine is also conventional. In general, one of the pastes incorporates the radical initiator and stabilizer (generally a peroxide) and the other paste, the accelerator, such as an amine like toluidine. Curing begins by simply mixing the two pastes together.

Photoinitiation or photocuring is another catalysis system to provide cured or hardened fillers or restoratives of the present invention. In this catalytic system, cured or hardened products are provided by exposure to actinic light of a suitable wavelength. Both ultraviolet and visible photocuring systems are known for use in restorative surgery and dentistry and any such system may be used in this invention. Exemplary systems are described in U.S. Pat. No. 4,110,184 to Dart et al., U.S. Pat. No. 4,698,373 to Tateosian et al., U.S. Pat. No. 4,491,453 to Koblitz et al., and U.S. Pat. No. 4,801,528 to Bennett.

A particularly useful system employs visible light curing, thus avoiding the potential danger inherent in curing with ultraviolet radiation. Visible light curing has been used in the dental field, and may also be applied to restorations of bony tissues. Quinones, as a class, find wide utility as photochemical initiators for visible light sensitizing systems, preferably when mixed with tertiary amines. It is preferred that an alpha diketone (quinone) such as camphoroquinone or biacetyl be mixed with an amine reducing agent such as n-alkyl dialkanolamine or trialkanolamine.

While embodiments of the present invention include biocompatible particles and a polymerizable mixture, other materials may be included in such compositions. Additional materials include radiopacifying agents, surface active agents, handling agents, pigments and reinforcing materials such as fibers. These materials are typically used in the systems described in this application and how these materials would be used would be understood by those of ordinary skill in this field.

Those of ordinary skill in the art will appreciate that the amount of particles used in conjunction with the polymerizable embodiments of the present invention will depend upon several variables including the identity of the polymerizable mixture, the particles and the particle sizes. For a particular composition, the choice of particles and particle size are selected to provide a desired viscosity, workability, ease of blending and biocompatibility.

In practice, light curable, hardenable compositions are provided in unitary form, e.g. they need not be mixed just prior to use. They are applied to the site for restoration and then exposed to visible light of a suitable wavelength to cause polymerization due to the catalyst system. Light sources and methods of application are well known to those of ordinary skill in the art and may be used with the compositions of the present invention. Thus, a composition of the present invention is either blended together from two “pastes”, if a thermal curing system is selected, or is used as provided, if a photocuring system is selected. The composition is applied to a prepared site for restoration. The restorative may then be smoothed or shaped into place and the material allowed to harden either through the passage of time, in the case of a thermal curing material or through application of actinic radiation in the case of a photocuring material. After initial polymerization and resultant hardening has occurred, the restoration becomes relatively strong. It will be load bearing and capable of supporting underlying and overlying structures within the body portion thus restored.

The autogenous bone used in embodiments of the present invention may be harvested from any suitable donor or donor site. For example, the autogenous bone may be harvested from one or more cadavers, processed and stored for later application to a patient. Alternatively, the autogenous bone may be harvested directly from a donor site of the patient, including the hip bone, pelvic bone, femur and iliac crest for immediate application. Autogenous bone is particularly suitable when used in accordance with the present invention because it possesses favorable growth and strength characteristics, including providing scaffolding for osteoconduction. Prior to application, the autogenous bone is combined with a suitable amount of the detectable particles of the present invention. The biocompatible detectable particles used in accordance with the present invention may provide increased mechanical strength to the application site.

The compositions of the present invention may be used in a variety of applications and may be delivered at, onto and/or into a site in a variety of ways. For example, once an implantation sight has been prepared, syringe delivery of a low-viscosity composition of the present invention may be expressed to fill the void. Optionally, the injection may be percutaneous. If desired, an implant may be used in conjunction with compositions of the present invention, such an implant being either metal, gutta percha, ceramic, polymer, or cured composition of this invention. Subsequent setting of the curable compositions of the present invention yields a restoration suitable for finishing or further restorative application.

The curable compositions of this invention are suitable, for example, to repair comminuted fractures. In this procedure, a traumatic injury has led to crushed or fragmented bone and a non-load bearing graft material would not be useful for its repair. At present, the use of metal plates and rods is a viable, but not completely satisfactory, option. The present invention may be used to reassemble bone fragments since the present compositions may be formulated into putty or paste for this purpose. Alternatively, photocuring materials may be used to cause tackification and curing in a short period of time to facilitate the reassembly of such fractured segments. It is also possible to employ hardened materials in accordance with the present invention, or traditional metal or ceramic bones, pins, plates, and the like for such restorations. The rapid load bearing capability of the materials of the present invention along with their bioactivity confer particular advantages to the present system.

The injectable embodiments of the present invention may be particularly suitable for treating bone diseases such as osteoporosis. Osteoporosis is a disease characterized by low bone mass and deterioration of bone tissue resulting in increased bone fragility and fracture, particularly to the hip, spine and wrist. For example, a painful condition often associated with osteoporosis is compression fractures of the vertebrae. The injectable compositions of the present invention may be injected onto or into the fractured tissue site to strengthen, repair and/or regenerate the fractured bone sites. One characteristic of this procedure is that it is minimally invasive, requiring a simple injection into the diseased site.

Embodiments of this invention also may be suitable for filling cavities created by excision of a bone cyst or other benign local destructive lesions. Prior to filling a cavity, the overlying cortex of the area is removed to create a window in the cavity. The contents of the cavity are removed and the walls are curetted thoroughly. The cavity is flushed with saline and the bone filler composition is then injected into the cavity until the cavity is completely filled.

Yet a further example of the utility of compositions of the present invention in orthopedics involving great stresses and procedural difficulties involves bipolar hip replacement or revision. In this procedure, implant fixation on the femoral stem side is simple, however, the acetabular cup attachment is very difficult, especially in revision cases. With the use of pins, the acetabulum may be used with compositions of the present invention to make up for lost bone of the acetabulum. Immediate function is important to this application and the load bearing ability of the present materials indicates it for such use. The biological bonding of the compositions of the present invention enhances strength and toughness in such procedures and prevents further absorption of existing bone.

1. A curable composition consisting essentially of biocompatible detectable composite particles having an exposed surface of carbon and a polymerizable mixture.

2. The composition of claim 1 wherein the polymerizable mixture comprises a curable acrylate/catalyst system.

3. The composition of claim 1 wherein the polymerizable mixture comprises a polymethylmethacrylate/catalyst system.

4. The composition of claim 1 wherein the composition consists essentially of 10-40 wt. % composite particles.

5. The composition of claim 1 wherein the exposed surface of carbon is an exposed surface of isotropic pyrolytic carbon.

6. The composition of claim 1 wherein the composite particles consist of zirconium oxide and carbon.

7. The composition of claim 1 wherein the particles have a transverse crosssection dimension of about 10-300 microns.

8. The composition of claim 1 wherein the particles have a transverse crosssection dimension of about 50-120 microns.

9. The composition of claim 1 wherein the particles are radiopaque.

10. A method for bone filling or restoration comprising the steps of applying a curable composition consisting essentially of biocompatible detectable composite particles having an exposed surface of carbon and a polymerizable mixture to bony tissue and curing the composition to provide a load bearing hardened composition.

11. The method of claim 10 wherein the exposed surface of carbon is an exposed surface of isotropic pyrolytic carbon.

12. The method of claim 10 wherein the applying step comprises injecting the curable composition at a site.

13. A bone filler or restorative comprising a curable composition of claim 1.

14. A composition consisting essentially of biocompatible detectable composite particles having an exposed surface of carbon and autogenous bone.

15. The composition of claim 14 wherein the composition consists essentially of 10-40% composite particles.

16. The composition of claim 14 wherein the detectable composite particles are radiopaque.

17. The composition of claim 14 wherein the exposed surface of carbon is an exposed surface of isotropic pyrolytic carbon.

18. The composition of claim 14 wherein composite particles consist of zirconium oxide and carbon.

19. The composition of claim 14 wherein the composite particles consist of carbon.

20. The composition of claim 14 wherein the composite particles have a transverse cross-section of about 10-300 microns.

21. The composition of claim 14 wherein the composite particles have a transverse cross-section of about 50-120 microns.

22. A bone filler or restorative comprising the composition of claim 14.

23. A method of bone filling or restoration comprising the step of applying to a patient a composition consisting essentially of biocompatible detectable composite particles having an exposed surface of carbon and autogenous bone.

24. The method of claim 23 wherein the applying step comprises injecting the composition into a site.

25. The method of claim 23 further comprising the steps of harvesting the autogenous bone and combining the autogenous bone and the detectable composite particles.

26. The method of claim 23 wherein the exposed surface of carbon is an exposed surface of isotropic pyrolytic carbon.
